(19) United States
(12) Patent Application Publication
(10) Pub. No.: US 2017/0065372 A1
(43) Pub. Date: Mar. 9, 2017

(54) ORTHODONTIC APPLIANCES CONFIGURED TO RECEIVE AND TRANSMIT BIOLOGIC STIMULATION

(71) Applicant: James Mah, Las Vegas, NV (US)

(72) Inventor: James Mah, Las Vegas, NV (US)

(21) Appl. No.: 14/846,961

(22) Filed: Sep. 7, 2015

(51) Int. Cl. A61C 7/00; A61C 1/07; A61C 7/08

(52) U.S. Cl. CPC A61C 7/008; A61C 7/08; A61C 1/07; A61N 2005/0606

(57) ABSTRACT

An orthodontic appliance that is configured to receive and transmit stimulation to a patient's teeth to enhance movement of such teeth, which includes an aligner that is configured to be fittingly and reversibly applied to a patient's teeth. The aligner includes a first area that is adapted to receive and transmit stimulation to one or more targeted underlying teeth (to enhance movement of the targeted underlying teeth). The aligner also includes a second area that is not adapted to receive and transmit stimulation to one or more non-targeted underlying teeth (or is otherwise adapted to prevent at least some stimulation from being transmitted to non-targeted underlying teeth), to avoid or reduce movement of the non-targeted teeth.

### FIELD OF THE INVENTION

The field of the present invention relates to orthodontic appliances that are configured to receive and transmit biologic stimulation to a patient's teeth, for the purpose of facilitating the movement of such teeth.

### BACKGROUND OF THE INVENTION

Orthodontic tooth movement is a biologic process that involves remodeling of bone and periodontal tissues. The process is complex, involving many cell types, interactions, and biologic events. Collectively, such events occur over time and determine the rate of tooth movement. At present, the maximum rate of tooth movement is not known, although there are many approaches intended to accelerate such movement.

The desire for expedited tooth movement is appealing to both patients and clinicians. Approaches to accelerate tooth movement may be categorized as invasive or non-invasive. Invasive approaches involve surgical techniques, such as corticotomy, corticision, or osseous perforation. Non-invasive approaches may involve mechanical vibration, wavelengths of light, ultrasound, and lasers. A common goal of such techniques is to stimulate biologic processes that produce faster rates of tooth movement.

Non-invasive approaches are commonly delivered to a patient's teeth through a generic tray in an integral unit. However, a generic tray may not accommodate all patients, since dental arches differ considerably among patients in dimensions and morphology. In addition, the vibration or light stimulus is generally delivered to all of a patient's teeth—although some teeth will be moved more than others. One drawback with current stimuli devices and techniques is that the stimuli are often delivered to teeth that do not require or benefit from such stimuli (and, in fact, the deleterious effects of such “off target” stimuli are unknown). Similarly, the stimulus may be diluted over the teeth that are not the intended target, which unnecessarily results in more energy and power requirements to generate and deliver the stimuli for the targeted teeth.

As the following will demonstrate, the devices and methods of the present invention address many of these drawbacks with currently-available devices and methods for delivering non-invasive movement stimuli to teeth.

### SUMMARY OF THE INVENTION

According to certain aspects of the invention, an orthodontic appliance is provided that is configured to receive and transmit stimulation to a patient's teeth to enhance movement of such teeth. The orthodontic appliance will preferably include a polymeric aligner that is configured to be fittingly and reversibly applied to a patient's teeth (e.g., the aligner may be produced to match the patient's dentition at the time of use). The aligner includes a first area that is adapted to receive and transmit stimulation to one or more targeted underlying teeth (to enhance movement of the targeted underlying teeth). In addition, the aligner will include a second area (outside of the first area) that is not adapted to receive and transmit stimulation to one or more non-targeted underlying teeth—or is otherwise adapted to actually prevent at least some stimulation from being transmitted to non-targeted underlying teeth, to avoid or reduce movement of the non-targeted teeth.

The invention provides that the stimulation is preferably a non-invasive stimulation, such as mechanical vibration, light energy, ultrasound energy, and/or laser energy. In certain preferred embodiments, the invention provides that the stimulation is mechanical vibration. In such embodiments, the first area of the aligner will include an exterior surface that is configured to enhance engagement (i.e., increase the frictional force or mechanical connection) between the first area of the aligner and a device that provides mechanical vibration to the targeted underlying teeth. For example, the invention provides that the exterior surface of the first area of the aligner may include ridges, a textured surface, or a protruding element or aperture that is adapted to be connected to a corresponding aperture or protruding element of the device that provides mechanical vibration.

In other embodiments, the stimulation may be light energy. In such embodiments, the first area of the aligner will preferably be transparent (or substantially transparent) to enhance an amount of light (stimulation) that is permitted to traverse the first area and contact one or more targeted underlying teeth. In addition, in such embodiments, the second area of the aligner is substantially opaque, to prevent an amount of light from traversing the second area and contacting one or more non-targeted underlying teeth. In certain related embodiments, the first area of the aligner may include an interior passage or path, which is configured to selectively channel the light energy through the aligner and to the targeted underlying teeth.

According to additional aspects of the invention, a set of orthodontic appliances is provided, with each appliance within the set being configured to receive and transmit stimulation to a patient's teeth to enhance movement of such teeth (as described in the embodiments above). In such embodiments, each orthodontic appliance within the set of appliances is configured to be fittingly and reversibly applied to the patient's teeth at different points in time during a treatment protocol, with each appliance having its own first area that is configured to receive and transmit biologic stimulation to the underlying teeth. In other words, each appliance will be custom made to the patient's dentition, at the applicable time during the treatment protocol (with the first area being located and oriented to deliver stimulation in accordance with the clinician's treatment protocol).

According to yet further aspects of the invention, methods for imparting movement to one or more teeth of a patient are provided. The methods generally comprising applying each appliance (aligner) within a set of orthodontic appliances to a patient's teeth according to a treatment protocol (over time), and further applying the desired stimulation to the first area of each aligner as described herein.

The above-mentioned and additional features of the present invention are further illustrated in the Detailed Description contained herein.

### BRIEF DESCRIPTION OF THE FIGURES

FIG. 1: a diagram of a first area of an aligner (located in the buccal side of the aligner) that includes an exterior surface that is configured to enhance engagement between the first area of the aligner and a device that provides mechanical vibration to the targeted underlying teeth.

FIG. 2: a diagram of a first area of an aligner (located in the occlusal side of the aligner) that includes an exterior surface that is configured to enhance engagement between the first area of the aligner and a device that provides mechanical vibration to the targeted underlying teeth.

FIG. 3: a diagram of a first area of the aligner that is substantially transparent to enhance an amount of light or laser energy that is permitted to traverse the first area and contact one or more targeted underlying teeth (the buccal side of the teeth).

FIG. 4: a diagram of a first area of an aligner that is adapted to receive non-invasive stimulation in the form of light or laser energy, with the first area being configured to channel the light or laser energy to the buccal side of the targeted underlying teeth.

FIG. 5: a flow diagram that summarizes the methods described herein for imparting movement to one or more teeth of a patient.

### DETAILED DESCRIPTION OF THE INVENTION

The following will describe in detail several preferred embodiments of the present invention. These embodiments are provided by way of explanation only, and thus, should not unduly restrict the scope of the invention. In fact, those of ordinary skill in the art will appreciate upon reading the present specification and viewing the present drawings that the invention teaches many variations and modifications, and that numerous variations of the invention may be employed, used, and made without departing from the scope and spirit of the invention.

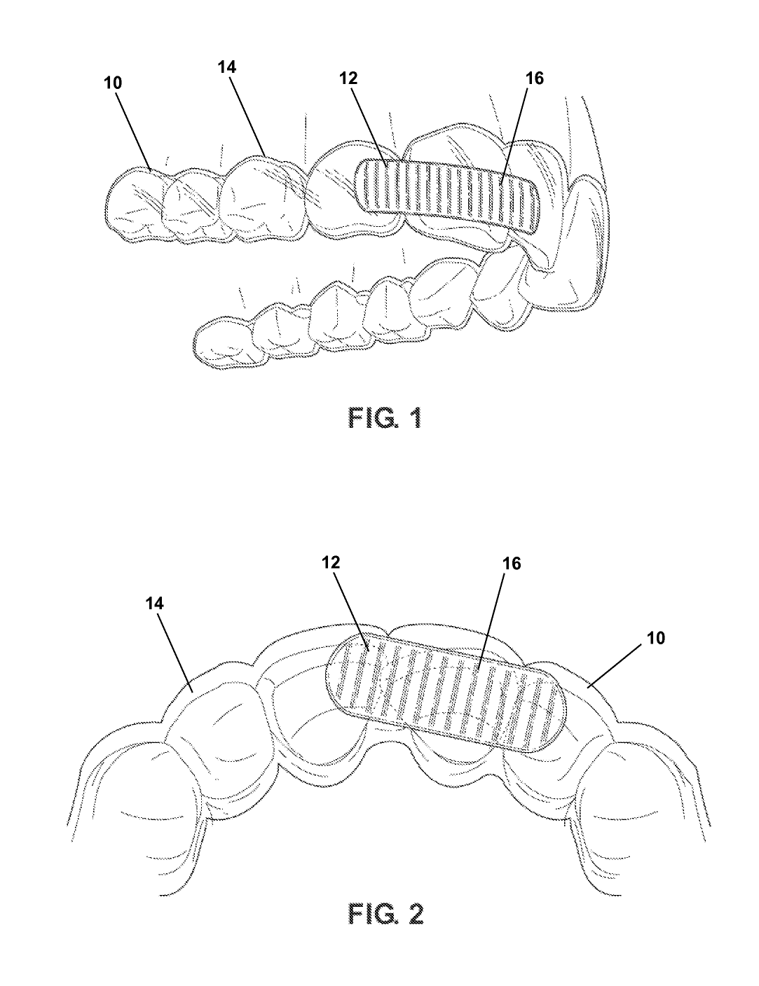

Referring now to FIGS. 1-5, according to certain preferred embodiments, an orthodontic appliance is provided that is configured to receive and transmit stimulation to a patient's teeth to enhance movement of such teeth. The invention provides that the stimulation will be effective to initiate certain biologic processes that have been shown to accelerate rates of tooth movement. The invention provides that the orthodontic appliance will preferably consist of (or include) an aligner 10 that is configured to be fittingly and reversibly applied to a patient's teeth (e.g., the aligner 10 is preferably produced to match the patient's dentition at the time of use and stimulation).

The aligner 10 may be created based on a dental cast, dental impression, or digital image of the patient's dentition. For example, in certain embodiments, the aligner 10 may be produced based on a dental mold or cast of a patient's dentition, which is created based on a dental impression (negative imprint) of the patient's teeth, which may be formed using materials such as polyvinylsiloxane (PVS) or alginate (which is applied to a patient's teeth in liquid or semi-solid form using an impression tray, and subsequently allowed to solidify). In such examples, the aligner 10 may be produced by thermoforming a polymeric aligner 10 around the dental mold, such that the resulting polymeric aligner 10 may then be fittingly and reversibly applied to the corresponding patient's teeth. In other embodiments, the aligner 10 may be created based on just the dental impression. In still other preferred embodiments, the aligner 10 may be created using a digital image of the patient's dentition and manufactured using stereolithography techniques. In such embodiments, the aligners 10 may be constructed in layers using a photo-sensitive liquid resin that subsequently cures into a hard thermoplastic material after being exposed to laser energy.

The invention provides that the aligner 10 includes a first area 12 that is adapted to receive and transmit non-invasive stimulation to one or more targeted underlying teeth (to enhance movement of the targeted underlying teeth). In addition, the aligner 10 includes a second area 14 that is not adapted to receive and transmit stimulation to one or more non-targeted underlying teeth—or is otherwise adapted to actually prevent at least some stimulation from being transmitted to non-targeted underlying teeth, to avoid or reduce movement of the non-targeted teeth. More particularly, the invention provides that the area of the appliance (aligner 10) that is adapted to receive stimulus may be selectively located to specific teeth (and, moreover, to specific areas of underlying teeth). As such, the aligner 10 will be configured to transmit stimulation to, and induce certain movements of, only targeted teeth (and will facilitate certain movements that are generally difficult to achieve, such as bodily translations). In certain embodiments, the invention provides that areas outside of the first area 12 (i.e., the second area 14) and other regions of the appliance may be insulated or isolated from receiving the applied stimuli.

According to certain preferred embodiments, the invention provides that the stimulation is a non-invasive stimulation, such as mechanical vibration, light energy, ultrasound energy, and/or laser energy. Referring now to FIGS. 1 and 2, in certain preferred embodiments, the invention provides that the stimulation is mechanical vibration. In such embodiments, the first area 12 of the aligner 10 will include an exterior surface 16 that is configured to enhance engagement (i.e., increase the frictional force or mechanical connection) between the first area 12 of the aligner 10 and a device that provides mechanical vibration to the targeted underlying teeth. Devices that are configured to provide mechanical vibration to underlying teeth are known in the art, and are typically used to provide pulses of vibration to an entire set of teeth through an integrally-connected/standardized tray (which, as mentioned above, suffers from several drawbacks, such as delivering stimuli to non-targeted teeth).

More specifically, for example, the invention provides that the exterior surface 16 of the first area 12 of the aligner 10 may include ridges, cross-hatches, a textured surface, or a protruding element or aperture that is adapted to be connected to a corresponding aperture or protruding element of the device that provides mechanical vibration. The invention provides that the first area 12 may be located on any surface of the aligner 10, such as the occlusal, buccal, labial, or lingual surfaces. Still further, the invention provides that the first area 12 may be integrally formed into the aligner 10 (e.g., during a thermoforming process that is used to create the aligner 10) or, alternatively, the first area 12 may be created separately and subsequently affixed to the desired area of the aligner 10 (e.g., using adhesives or mechanical attachment means). In the latter case, the first area 12 may be manufactured using certain rigid materials, e.g., stainless steel, which exhibit an enhanced ability to receive, withstand, and transmit mechanical stimuli to underlying teeth.

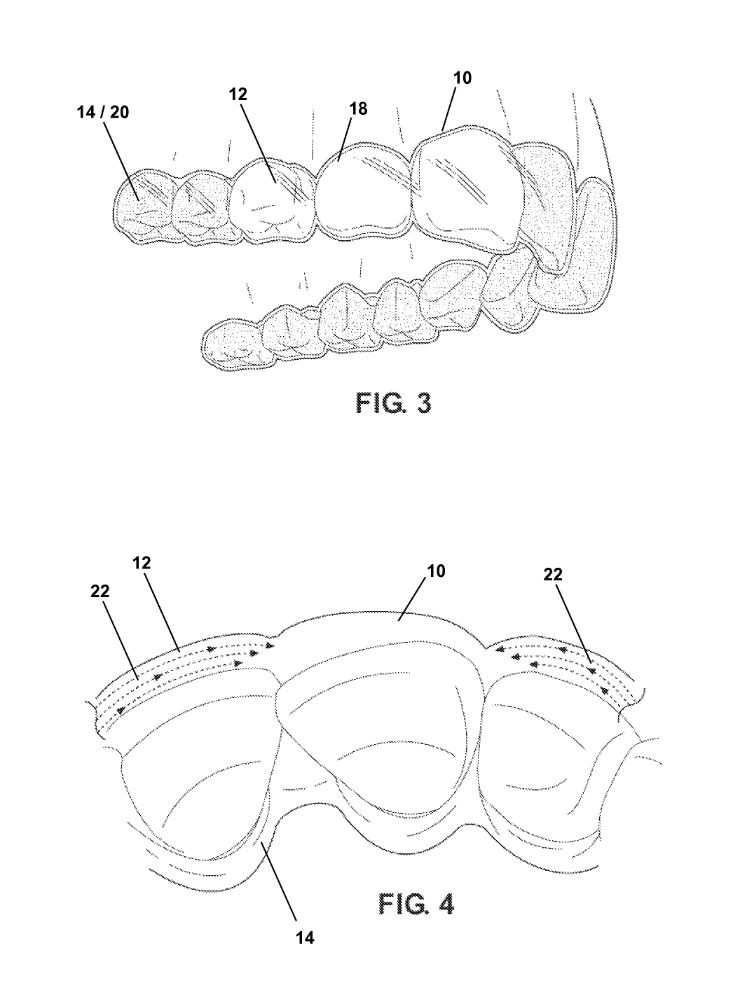

Referring now to FIG. 3, in other embodiments, the stimulation may be light (or laser) energy. In such embodiments, the first area 12 of the aligner 10 will preferably be transparent 18 (or substantially transparent) to enhance an amount of light or laser (stimulation) that is permitted to traverse the first area 12 and contact one or more targeted underlying teeth. More specifically, the invention provides that the first area 12 of the aligner 10 will be optically clear (or may consist of a removed section of the aligner 10), to allow light or laser transmission without interference. In addition, the invention provides that the material used to fabricate the first area 12 of the aligner 10 may be optimized to allow for maximum transmission of energy from sources of light or laser energy. Such materials may include grain boundaries or planes within the material to efficiently transmit and direct the energy along a specified direction.

In addition, in such embodiments, the second area 14 of the aligner may be substantially opaque 20, to prevent an amount of light or laser energy from traversing the second area 14 and contacting one or more non-targeted underlying teeth. The second area 14 of the aligner may be substantially opaque 20 by texturing (or imparting a rough surface to) such areas of the aligner 10. In addition, the second area 14 of the aligner may be coated with materials to prevent light or laser penetration, e.g., using tin foils, acrylics, and solid particles that are effective to prevent transmission of light or laser energy. The invention further provides that, although the Figures of the present application illustrate an aligner 10 that is trimmed at a patient's gum line, the aligner 10 may be extended towards the root apices of the teeth as well (if desired), to optically block regions of a tooth (or set of teeth) and bone that should not be stimulated.

Referring now to FIG. 4, according to yet further embodiments of the present invention, the first area 12 of the aligner 10 may include an interior passage or path 22, which is configured to selectively channel light or laser energy to the targeted underlying teeth. Such embodiments may be particularly useful to impart certain rotational movements to a targeted tooth (or set of teeth). In such embodiments, the interior passage or path 22 of the aligner 10 will preferably be formed using a material that is effective to channel the light or laser energy to the targeted underlying teeth, such as using fiber optics, laminated materials, tin foils, acrylics, or nanoparticles that are effective to prevent the light or laser energy from escaping the passage or path 22 into non-target areas, and will facilitate the focused delivery of the light or laser energy to the targeted underlying teeth. Such embodiments are particularly beneficial insofar as they allow stimulation to be applied to targeted underlying teeth at a desired direction and orientation to stimulate tooth movement, e.g., light or laser energy may even be channeled towards a root, surrounding bone, and/or surrounding gum tissues. Still further, in such embodiments, the aligner 10 may include an exterior port, which is adapted to receive a device that delivers the light or laser energy to the targeted underlying teeth via the passage or path 22.

According to additional embodiments of the present invention, a set of orthodontic appliances is provided. In such embodiments, each appliance within the set is configured to receive and transmit stimulation to a patient's teeth to enhance movement of such teeth (as described in the embodiments above). In such embodiments, each orthodontic appliance within the set of appliances is configured to be fittingly and reversibly applied to the patient's teeth at different points in time during a treatment protocol (and the location of each first area 12 within each aligner 10 is designed to impart stimulation at the desired location within the patient's dentition). In other words, each appliance will be custom made to the patient's dentition (as described above), at the applicable time during the treatment protocol, and used to receive and transmit stimulation to the patient's underlying teeth as necessary to carry out the desired treatment protocol.

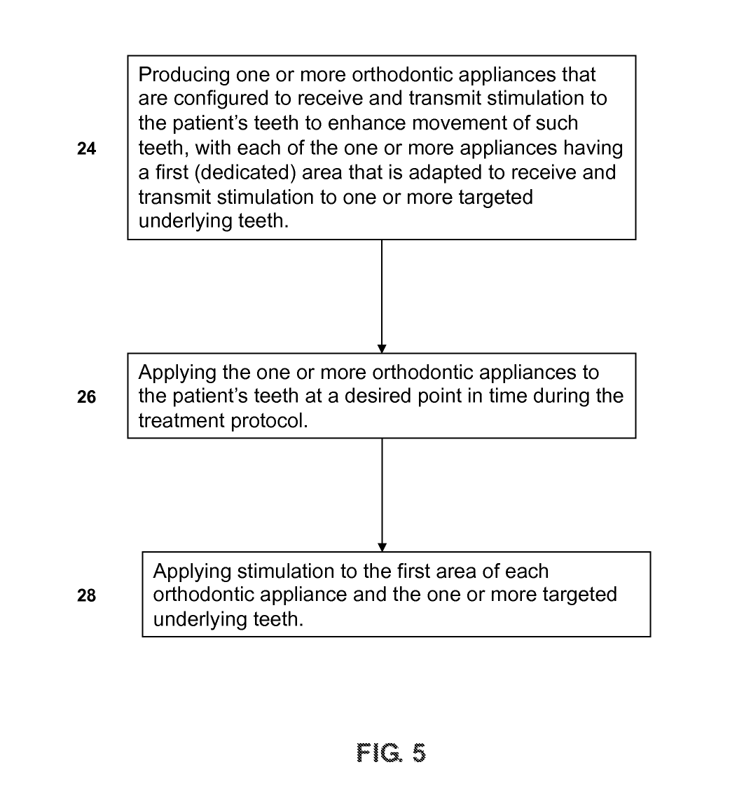

Referring now to FIG. 5, according to yet further embodiments of the invention, methods for imparting movement to one or more teeth of a patient are provided. The methods generally include applying each appliance (aligner 10) within a set of orthodontic appliances to a patient's teeth according to a treatment protocol, and further applying the desired stimulation to the first area 12 of each aligner 10 as described herein. More specifically, the methods of the present invention include the steps of 24 producing a set of orthodontic appliances (aligners 10) that are configured to receive and transmit stimulation to the patient's teeth to enhance movement of such teeth, with each orthodontic appliance within the set of orthodontic appliances including an aligner 10 that is configured to be fittingly and reversibly applied to the patient's teeth at a different point in time during a treatment protocol. In such embodiments, as described herein, a first area 12 of each aligner 10 is adapted to receive and transmit stimulation to one or more targeted underlying teeth, to enhance movement of the targeted underlying teeth. A second area 14 of each aligner 10 will not be adapted to receive and transmit stimulation to one or more non-targeted underlying teeth (or is otherwise adapted to prevent at least some stimulation from being transmitted to non-targeted underlying teeth, to avoid or reduce movement of the non-targeted teeth).

The methods of the present invention further include the steps of 26 applying each orthodontic appliance (aligner 10) within the set of orthodontic appliances to the patient's teeth at a desired point in time during the treatment protocol and 28 applying stimulation to the first area 12 of each aligner 10 and the one or more targeted underlying teeth. As described above, in these embodiments, the stimulation is preferably a non-invasive stimulation, such as mechanical vibration, light energy, ultrasound energy, or laser energy. The stimuli may vary in intensity, duration, and frequency—as selected by the clinician. Additionally, in the case of mechanical stimuli, the stimuli may be generated by a battery powered device or manually (via a tooth brush or other means of generating a mechanical vibration). In many orthodontic treatment protocols, not all teeth should be moved at once. The aligners 10 and methods described herein enable a clinician to facilitate movement to only certain targeted teeth (while simultaneously withholding movement-inducing stimuli from other surrounding teeth). As a patient's teeth change during a treatment protocol, a new aligner 10 may be produced and used as described herein, to stimulate the movement of additional teeth according to the clinician's recommended treatment protocol.

The many aspects and benefits of the invention are apparent from the detailed description, and thus, it is intended for the following claims to cover all such aspects and benefits of the invention which fall within the scope and spirit of the invention. In addition, because numerous modifications and variations will be obvious and readily occur to those skilled in the art, the claims should not be construed to limit the invention to the exact construction and operation illustrated and described herein. Accordingly, all suitable modifications and equivalents should be understood to fall within the scope of the invention as claimed herein.

What is claimed is:

1. An orthodontic appliance that is configured to receive and transmit stimulation to a patient's teeth to enhance movement of said teeth, which comprises an aligner that is configured to be fittingly and reversibly applied to a patient's teeth, wherein:
(i) a first area of the aligner is adapted to receive and transmit stimulation to one or more targeted underlying teeth, to enhance movement of the targeted underlying teeth; and
(ii) a second area of the aligner is not adapted to receive and transmit stimulation to one or more non-targeted underlying teeth or is adapted to prevent at least some stimulation from being transmitted to non-targeted underlying teeth, to avoid or reduce movement of the non-targeted teeth.

2. The orthodontic appliance of claim 1, wherein the stimulation is a non-invasive stimulation.

3. The orthodontic appliance of claim 2, wherein the stimulation is selected from the group consisting of mechanical vibration, light energy, ultrasound energy, and laser energy.

4. The orthodontic appliance of claim 3, wherein (i) the stimulation is mechanical vibration and (ii) the first area of the aligner comprises an exterior surface that is configured to enhance engagement between the first area of the aligner and a device that provides mechanical vibration to the targeted underlying teeth.

5. The orthodontic appliance of claim 4, wherein the exterior surface of the first area of the aligner comprises ridges; a textured surface; or a protruding element or aperture that is adapted to be connected to a corresponding aperture or protruding element of the device that provides mechanical vibration.

6. The orthodontic appliance of claim 3, wherein (i) the stimulation is light or laser energy; (ii) the first area of the aligner is substantially transparent to enhance an amount of light or laser energy that is permitted to traverse the first area and contact one or more targeted underlying teeth; and (iii) the second area of the aligner is substantially opaque to prevent an amount of light or laser energy from traversing the second area and contacting one or more non-targeted underlying teeth.

7. The orthodontic appliance of claim 3, wherein (i) the stimulation is light or laser energy and (ii) the first area of the aligner is configured to selectively channel the light or laser energy to the targeted underlying teeth.

8. A set of orthodontic appliances that are configured to receive and transmit stimulation to a patient's teeth to enhance movement of said teeth, wherein each orthodontic appliance within the set of orthodontic appliances comprises an aligner that is configured to be fittingly and reversibly applied to a patient's teeth at a different point in time during a treatment protocol, wherein:
(i) a first area of each aligner is adapted to receive and transmit stimulation to one or more targeted underlying teeth, to enhance movement of the targeted underlying teeth; and
(ii) a second area of each aligner is not adapted to receive and transmit stimulation to one or more non-targeted underlying teeth or is adapted to prevent at least some stimulation from being transmitted to non-targeted underlying teeth, to avoid or reduce movement of the non-targeted teeth.

9. The set of orthodontic appliances of claim 8, wherein the stimulation is a non-invasive stimulation.

10. The set of orthodontic appliances of claim 9, wherein the stimulation is selected from the group consisting of mechanical vibration, light energy, ultrasound energy, and laser energy.

11. The set of orthodontic appliances of claim 10, wherein (i) the stimulation is mechanical vibration and (ii) the first area of each aligner comprises an exterior surface that is configured to enhance engagement between the first area of each aligner and a device that provides mechanical vibration to the targeted underlying teeth.

12. The set of orthodontic appliances of claim 11, wherein the exterior surface of the first area of each aligner comprises ridges; a textured surface; or a protruding element or aperture that is adapted to be connected to a corresponding aperture or protruding element of the device that provides mechanical vibration.

13. The set of orthodontic appliances of claim 10, wherein (i) the stimulation is light or laser energy; (ii) the first area of each aligner is substantially transparent to enhance an amount of light or laser energy that is permitted to traverse the first area and contact one or more targeted underlying teeth; and (iii) the second area of each aligner is substantially opaque to prevent an amount of light or laser energy from traversing the second area and contacting one or more non-targeted underlying teeth.

14. The set of orthodontic appliances of claim 10, wherein (i) the stimulation is light or laser energy and (ii) the first area of each aligner is configured to selectively channel the light or laser energy to the targeted underlying teeth.

15. A method for imparting movement to one or more teeth of a patient, which comprises:
(a) producing a set of orthodontic appliances that are configured to receive and transmit stimulation to the patient's teeth to enhance movement of said teeth, wherein each orthodontic appliance within the set of orthodontic appliances comprises an aligner that is configured to be fittingly and reversibly applied to the patient's teeth at a different point in time during a treatment protocol, wherein:
(i) a first area of each aligner is adapted to receive and transmit stimulation to one or more targeted underlying teeth, to enhance movement of the targeted underlying teeth; and
(ii) a second area of each aligner is not adapted to receive and transmit stimulation to one or more non-targeted underlying teeth or is adapted to prevent at least some stimulation from being transmitted to non-targeted underlying teeth, to avoid or reduce movement of the non-targeted teeth;
(b) applying each orthodontic appliance within the set of orthodontic appliances to the patient's teeth at a desired point in time during the treatment protocol; and
(c) applying stimulation to the first area of each aligner and the one or more targeted underlying teeth, when each orthodontic appliance is applied to the patient's teeth in step (b).

16. The method of claim 15, wherein the stimulation is a non-invasive stimulation.

17. The method of claim 16, wherein the stimulation is selected from the group consisting of mechanical vibration, light energy, ultrasound energy, and laser energy.

18. The method of claim 17, wherein (i) the stimulation is mechanical vibration and (ii) the first area of each aligner comprises an exterior surface that is configured to enhance engagement between the first area of each aligner and a device that provides mechanical vibration to the targeted underlying teeth.

19. The method of claim 18, wherein the exterior surface of the first area of each aligner comprises ridges; a textured surface; or a protruding element or aperture that is adapted to be connected to a corresponding aperture or protruding element of the device that provides mechanical vibration.

20. The method of claim 17, wherein (i) the stimulation is light or laser energy; (ii) the first area of each aligner is substantially transparent to enhance an amount of light or laser energy that is permitted to traverse the first area and contact one or more targeted underlying teeth; and (iii) the second area of each aligner is substantially opaque to prevent an amount of light or laser energy from traversing the second area and contacting one or more non-targeted underlying teeth.
